(19) United States
(12) Patent Application Publication
(10) Pub. No.: US 2021/0322002 A1
(43) Pub. Date: Oct. 21, 2021

(54) SURGICAL INSTRUMENT FOR PERFORMING A PURSE STRING SUTURE

(71) Applicant: Covidien LP, Mansfield, MA (US)

(72) Inventors: Jacob C. Baril, Norwalk, CT (US); Garrett P. Ebersole, Hamden, CT (US); Justin Thomas, New Haven, CT (US); Saumya Banerjee, Hamden, CT (US); Matthew A. Dinino, Newington, CT (US); Roy J. Pilletere, North Haven, CT (US)

(21) Appl. No.: 16/850,548

(22) Filed: Apr. 16, 2020

(51) Int. Cl. A61B 17/04; A61B 17/06

(52) U.S. Cl. CPC A61B 17/0469; A61B 2017/1142; A61B 17/06166

(57) ABSTRACT

A surgical instrument includes a handle assembly, an elongated portion, a first jaw member, a second jaw member, and a needle assembly. Each jaw member includes a plurality of undulations and a needle channel. Each undulation includes a peak, a valley and a connecting portion interconnecting one peak with one valley. Each needle channel extends through each undulation of the respective jaw member and is parallel to a longitudinal axis defined by the elongated portion. The needle assembly includes a first needle and a second needle. Each of the first needle and the second needle includes a suture catch configured to releasably engage a portion of a suture. Each needle is movable from a first position where a distal end of the needle is positioned proximally of the respective jaw member, to a second position where the distal end of the needle is positioned distally of the respective jaw member.

### BACKGROUND

### Technical Field

The present disclosure relates generally to a surgical instrument for applying a suture to body tissue. More particularly, the present disclosure relates to a surgical instrument suitable for laparoscopically applying a purse-string style suture around hollow tissue organs.

### Background of Related Art

Anastomosis is the surgical joining of separate hollow organ sections. Typically, an anastomosis procedure follows surgery in which a diseased or defective section of hollow tissue is removed, and the end sections are stapled via a linear surgical stapler. Depending on the desired anastomosis procedure, the end sections may be joined by either circular, end-to-end, or side-to-side organ reconstruction methods.

In a circular anastomosis procedure, the two ends of the organ sections are joined by means of a stapling instrument which is inserted through a first opening in a patient. The stapling instrument drives a circular array of staples through the end section of each organ section and simultaneously cores any tissue interior of the driven circular array of staples to free the tubular passage. Typically, these instruments include an elongated shaft having a handle portion at a proximal end to actuate the instrument and a staple holding component disposed at a distal end. An anvil assembly including an anvil rod with attached anvil head is mounted to the distal end of the instrument adjacent the staple holding component. The anvil assembly is then positioned within a distal portion of the hollow organ, for instance. Next, the tissue of the tubular vessel is tied or purse stringed with a suture about a portion of the anvil assembly. Then, opposed end portions of tissue of the hollow organ(s) clamped between the anvil head and the staple holding component, the clamped tissue is stapled, and finally the annular knife is advanced to core the tissue within the hollow organ.

During such surgical procedures, it is often challenging to apply the suture to the tissue about a portion of the anvil assembly laparoscopically. Accordingly, it may be helpful to provide a surgical instrument to facilitate the laparoscopic application of a purse string suture to an anvil assembly.

### SUMMARY

The present disclosure relates to a surgical instrument including a handle assembly, an elongated portion extending distally from the handle assembly and defining a longitudinal axis, an end effector, and a needle assembly. The end effector is disposed adjacent a distal end of the elongated portion and includes a first jaw member and a second jaw member. The first jaw member is movable relative to the second jaw member between a first position where the jaw members are in an open position and a second position where the jaw members are in an approximated position. The first jaw member includes a plurality of undulations and a needle channel. Each undulation of the plurality of undulations of the first jaw member includes a peak, a valley, and a connecting portion which interconnects one peak with one valley. The needle channel of the first jaw member extends through each undulation of the plurality of undulations of the first jaw member and is parallel to the longitudinal axis. The second jaw member includes a plurality of undulations and a needle channel. Each undulation of the plurality of undulations of the second jaw member includes a peak, a valley, and a connecting portion which interconnects one peak with one valley. The needle channel of the second jaw member extends through each undulation of the plurality of undulations of the second jaw member and is parallel to the longitudinal axis. The needle assembly includes a first needle and a second needle. Each of the first needle and the second needle includes a suture catch configured to releasably engage a portion of a suture. The first needle is movable from a first position where a distal end of the first needle is positioned proximally of the first jaw member to a second position where the distal end of the first needle is positioned distally of the first jaw member. The second needle is movable from a first position where a distal end of the second needle is positioned proximally of the second jaw member to a second position where the distal end of the second needle is positioned distally of the second jaw member.

In aspects, the peaks of at least a majority of undulations of the plurality of undulations of the first jaw member and the second jaw member may be flat along a majority of their lengths in a direction that is parallel to the longitudinal axis. The peaks of at least a majority of undulations of the plurality of undulations of the second jaw member may be flat along a majority of their lengths in a direction that is parallel to the longitudinal axis. In aspects, the valleys of at least a majority of undulations of the plurality of undulations of the first jaw member and the second jaw member may be flat along a majority of their lengths in a direction that is parallel to the longitudinal axis.

In additional aspects, a cumulative length of the flat portions of the peaks of the plurality of undulations of the first jaw member and the second jaw member may be between about 40% and about 60% of a total length of the respective jaw member.

In aspects, the connecting portions of at least a majority the plurality of undulations of the first jaw member may be perpendicular to the longitudinal axis.

In additional aspects, the needle channel of the first jaw member may extend through the connecting portion of each undulation of the plurality of undulations of the first jaw member, and the needle channel of the second jaw member may extend through the connecting portion of each undulation of the plurality of undulations of the second jaw member.

In aspects, each undulation of the plurality of undulations of the first jaw member may be longitudinally aligned with one undulation of the plurality of undulations of the second jaw member.

In further aspects, the needle channel of the first jaw member may define a cross-sectional shape having a first portion and a second portion. The first portion of the cross-sectional shape may be circular, and the second portion of the cross-sectional shape may be rectangular. The first portion of the cross-sectional shape of the needle channel of the first jaw member may extend through the connecting portion of each undulation of the plurality of undulations of the first jaw member. The second portion of the cross-sectional shape of the needle channel of the first jaw member may extend through the peak of each undulation of the plurality of undulations of the first jaw member.

The present disclosure also relates to a surgical instrument including a handle assembly, an elongated portion extending distally from the handle assembly and a defining a longitudinal axis, a first jaw member, and a needle channel. The first jaw member is disposed adjacent a distal end of the elongated portion and includes a plurality of undulations. Each undulation includes a peak, a valley, and a connecting portion which interconnects one peak with one valley. The needle channel extends through each undulation of the plurality of undulations and is parallel to the longitudinal axis. The needle channel defines a cross-section having a first portion and a second portion. The first portion extends through the connecting portion of each undulation of the plurality of undulations and the second portion extends through the peak of each undulation of the plurality of undulations.

In aspects, the first portion of the cross-section of the needle channel may be circular and the second portion of the cross-section of the needle channel may be rectangular.

In additional aspects, the peaks of at least a majority of undulations of the plurality of undulations of the first jaw member may be flat along a majority of their lengths in a direction that is parallel to the longitudinal axis, the valleys of at least a majority of undulations of the plurality of undulations of the first jaw member may be flat along a majority of their lengths in a direction that is parallel to the longitudinal axis, and the connecting portions of at least a majority of the plurality of undulations of the first jaw member may be perpendicular to the longitudinal axis.

In aspects, a cumulative length of the flat portions of the peaks of the plurality of undulations of the first jaw member may be between about 40% and about 60% of a total length of the first jaw member.

The present disclosure also relates to a method of performing a purse string closure, including positioning an end effector of a laparoscopic surgical instrument adjacent a surgical site, moving a first jaw member of the end effector toward a second jaw member of the end effector to clamp tissue therebetween, advancing a first needle and a suture through a plurality of undulations of the first jaw member such that the first needle punctures tissue disposed between adjacent undulations of the first jaw member, and advancing a second needle and the suture through a plurality of undulation of the second jaw member such that the second needle punctures tissue disposed between adjacent undulations of the second jaw member.

In aspects, advancing the first needle may include moving the first needle from a first position where a distal tip of the first needle is disposed proximally of the first jaw member to a second position where the distal tip of the first needle is disposed distally of the first jaw member.

In additional aspects, advancing the first needle and advancing the second needle may occur simultaneously.

### DESCRIPTION OF THE DRAWINGS

Various aspects of the present disclosure are illustrated herein with reference to the accompanying drawings, wherein:

FIG. 1 is a perspective view of a surgical instrument according to aspects of the present disclosure;

FIG. 2 is an enlarged view the area of detail indicated in FIG. 1 illustrating jaw members of the surgical instrument in an open position;

FIG. 3 is a perspective view of the jaw members of the surgical instrument of FIGS. 1 and 2 in an approximated position;

FIG. 4 is an assembly view of the jaw members, a needle assembly, and a suture of the surgical instrument of FIG. 2;

FIG. 5 is a side cross-sectional view of the jaw members of the surgical instrument in an open position taken along line 5-5 in FIG. 2, shown with the needle assembly, the suture, and with tissue between the jaw members;

FIG. 6 is an enlarged view of the area of detail indicated in FIG. 5;

FIG. 7 is a side cross-sectional view of the jaw members of the surgical instrument of FIG. 5 in the approximated position, and with tissue between the jaw members;

FIG. 8 is an end cross-sectional view of the portion of the surgical instrument, needle assembly, and suture taken along line 8-8 in FIG. 6;

FIG. 9 is an end cross-sectional view of the portion of the surgical instrument and tissue taken along line 9-9 in FIG. 7;

FIG. 10 is a side cross-sectional view of the jaw members of the surgical instrument of FIG. 7 in the approximated position about tissue, and with the needle assembly and the suture in an advanced position;

FIG. 11 is a schematic, view of tubular tissue that has been sutured with the surgical instrument of the present disclosure;

FIG. 12 is a side view of jaw members of a surgical instrument in an open position according to an additional aspect of the present disclosure; and

FIG. 13 is a side view of the jaw member of FIG. 12 in an approximated position.

### DETAILED DESCRIPTION

Aspects of the presently disclosed surgical instrument will now be described in detail with reference to the drawings wherein like numerals designate identical or corresponding elements in each of the several views. As is common in the art, the term “proximal” refers to that part or component closer to the user or operator, i.e. surgeon or physician, while the term “distal” refers to that part or component farther away from the user.

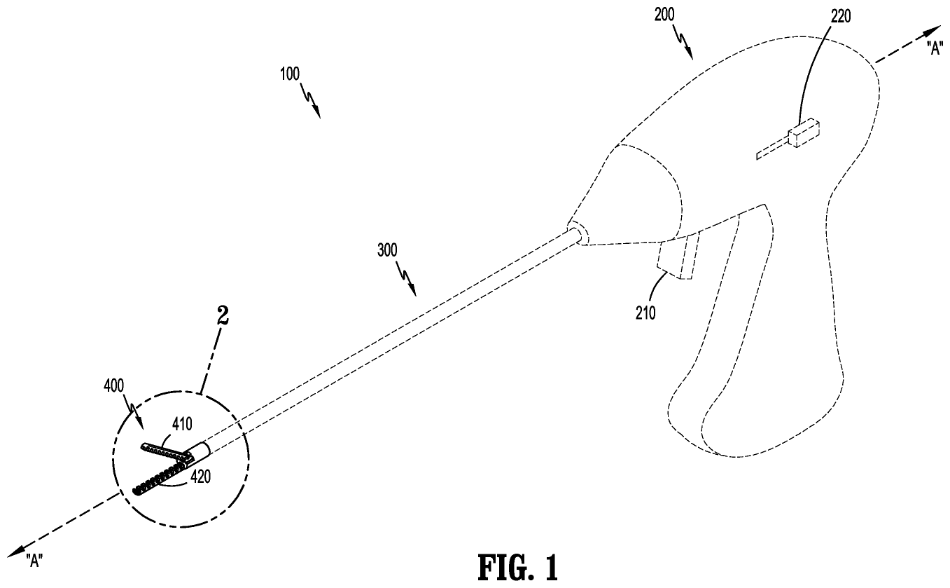

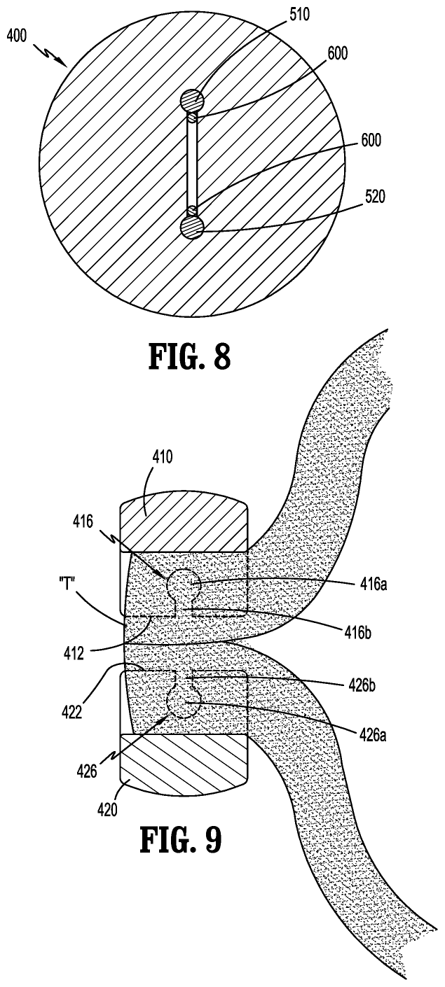

FIGS. 1-10 illustrate an exemplary surgical instrument according to the present disclosure, referenced generally as surgical instrument 100. With initial reference to FIG. 1, the surgical instrument 100 includes a handle assembly 200, an elongated portion 300 extending distally from the handle assembly 200 and defining a longitudinal axis “A-A,” and an end effector 400 disposed adjacent a distal end of the elongated portion and which includes a first jaw member 410 and a second jaw member 420.

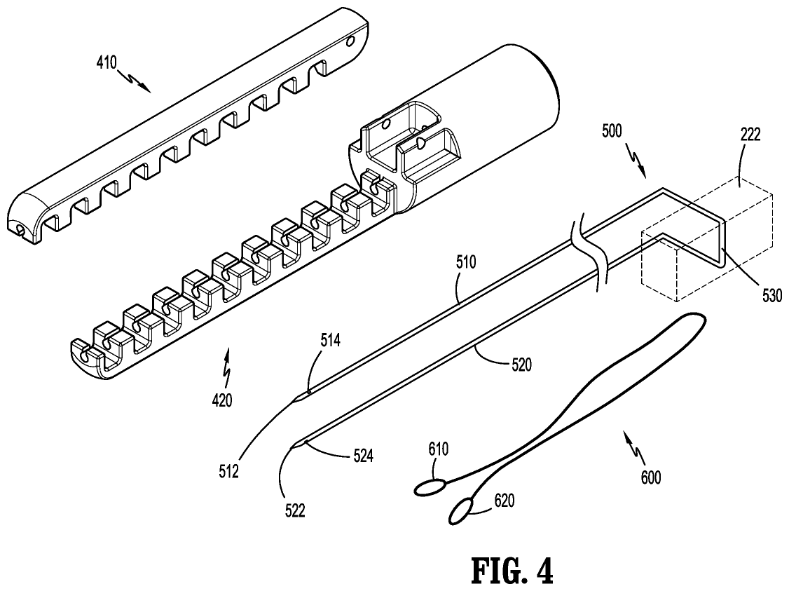

The handle assembly 200 includes a first actuator or trigger 210 and a second actuator or slider 220. Actuation of the trigger 210 is configured to translate a drive member longitudinally, which results in at least one jaw member (e.g., the first jaw member 410) to move with respect to the other jaw member (e.g., the second jaw member 420). That is, actuation of the trigger 210 causes the jaw members 410, 420 to move between an open position (e.g., FIGS. 1, 2, and 5) and an approximated position (e.g., FIGS. 3, 7, and 10). With particular reference to FIGS. 1 and 4, actuation of the slider 220 is configured to translate an elongated slide member 222 longitudinally (FIG. 4), which is mechanically coupled to a needle assembly 500 (FIG. 4).

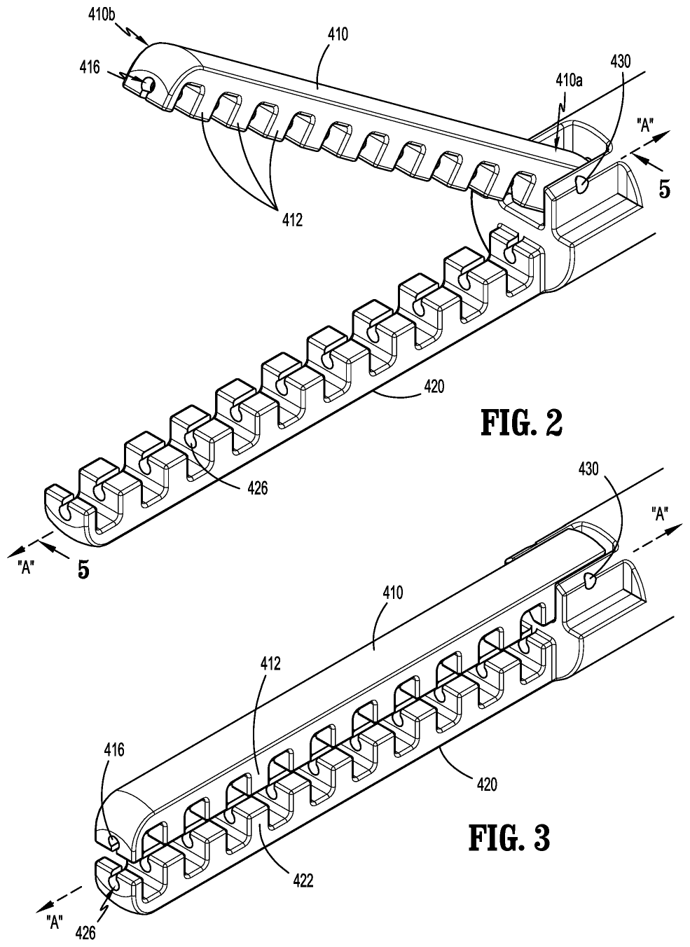

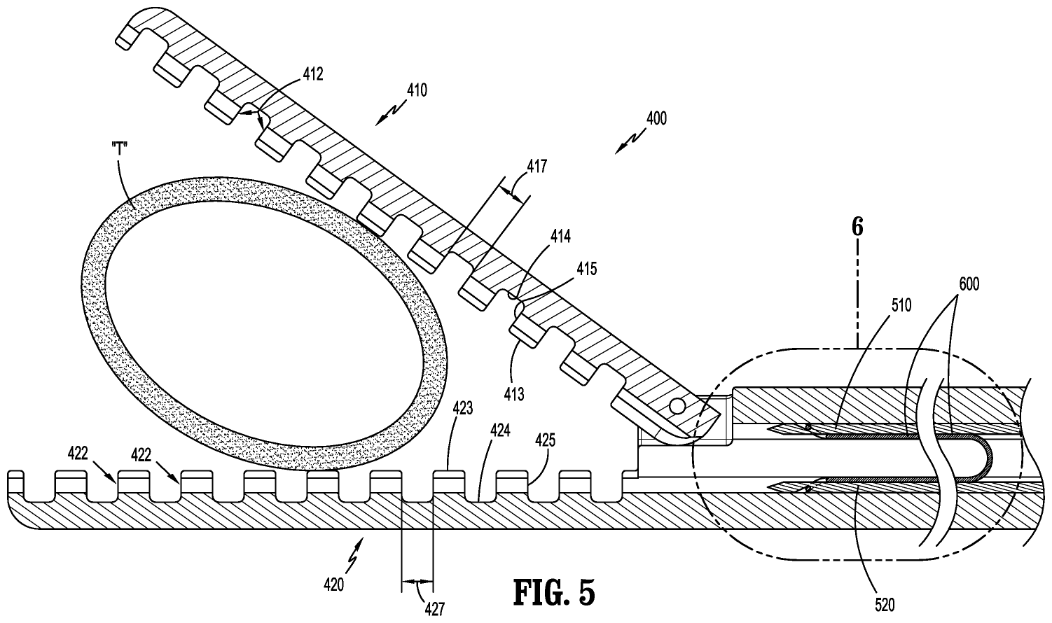

Details of the first jaw member 410 and the second jaw member 420 are shown in FIGS. 2-5 and 7. The first jaw member 410 is pivotable relative to the second jaw member 420 about a pivot point 430. The first jaw member 410 includes a plurality of undulations 412 extending between a proximal portion of the first jaw member 410 and a distal portion of the first jaw member 410. With particular reference to FIG. 5, each undulation 412 is defined by a flat or generally flat peak 413, a flat or generally flat valley 414, and a connecting portion 415 which interconnects one peak 413 with one valley 414. As shown, at least a majority of each connecting portion 415 is perpendicular or generally perpendicular to the peak, 413, the valley 414 and the longitudinal axis “A-A.” The term “at least a majority” as used herein means that more than half of the length of each connecting portion of each undulation is perpendicular or generally perpendicular to the peak, the valley, and the longitudinal axis. It should of course be understood that “at least a majority” also embraces the situation where the entire connecting portion of each undulation present in the device is perpendicular or generally perpendicular to the peak, the valley, and the longitudinal axis. Additionally, the second jaw member 420 includes a plurality of undulations 422 extending between a proximal portion of the second jaw member 420 and a distal portion of the second jaw member 420. Each undulation 422 is defined by a flat or generally flat peak 423, a flat or generally flat valley 424, and a connecting portion 425 which interconnects one peak 423 with one valley 424. As shown, at least a majority of the connecting portion 425 is perpendicular or generally perpendicular to the peak, 423, the valley 424 and the longitudinal axis “A-A.”

More particularly, each peak 413, 423 is flat or generally flat along a majority or an entirety of its length and is parallel to the longitudinal axis “A-A” (when the jaw members are in the approximated position). Further, cumulatively, the length of the flat portions of each of the peaks 413, 423 is in the range of about 40% to about 60% (e.g., about 50%) of the total length of the respective jaw member 410, 420. Additionally, each valley 414, 424 is flat or generally flat along a majority or an entirety of its length and is parallel to the longitudinal axis “A-A” (when the jaw members are in the approximated position). Further, cumulatively, the length of the flat portions of each of the valleys 414, 424 is in the range of about 40% to about 60% (e.g., about 50%) of the total length of the respective jaw member 410, 420.

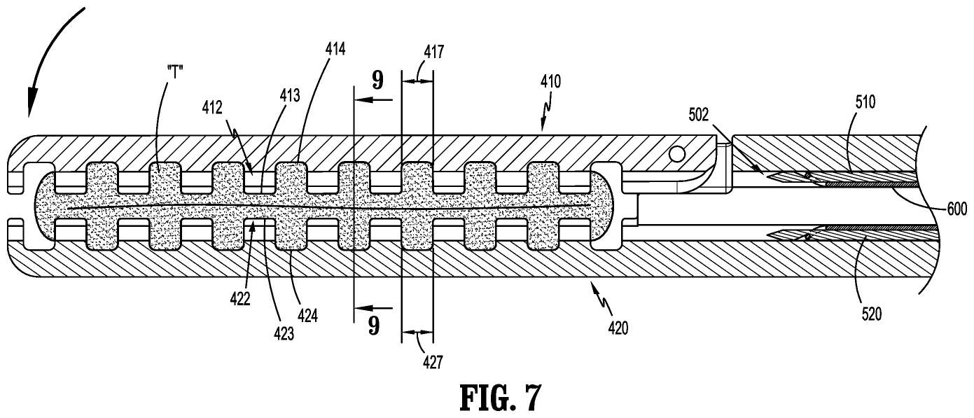

With particular reference to FIG. 7, each peak 413 and each valley 414 of the undulations 412 of the first jaw member 410 are aligned (e.g., longitudinally aligned) with a corresponding peak 423 and a corresponding valley 424 of the undulations 422 of the second jaw member 420. In the illustrated aspect, the first jaw member 410 and the second jaw member 420 include the same number of undulations 412, 422, respectively, as each other. In other aspects, the first jaw member 410 may have more or fewer undulations 412 than the second jaw member 420. Additionally, in the illustrated aspect, each of the first jaw member 410 and the second jaw member 420 includes ten valleys 414, 424, respectively. In other aspects, the first jaw member 410 and/or the second jaw member 420 includes more or fewer than ten valleys 414, 424.

Referring to FIGS. 2, 3, and 9, the first jaw member 410 includes a needle channel 416, and the second jaw member 420 includes a needle channel 426. The needle channel 416 of the first jaw member 410 is parallel to the longitudinal axis “A-A” and extends entirely through the first jaw member 410. The needle channel 426 of the second jaw member 420 is parallel to the longitudinal axis “A-A” and extends entirely through the second jaw member 420.

Further, and with particular reference to FIG. 9, the needle channel 416 of the first jaw member 410 defines a keyhole-like shape having a first portion 416a with a circular cross-section, and a second portion 416b with a rectangular cross-section. The first portion 416a of the needle channel 416 extends through each connection portion 415 of the undulations 412 of the first jaw member 410, and the second portion 416b of the needle channel 416 interconnects the first portion 416a of the needle channel 416 and each peak 413 of the first jaw member 410. Similarly, the needle channel 426 of the second jaw member 420 defines a keyhole-like shape having a first portion 426a with a circular cross-section, and a second portion 426b with a rectangular cross-section. The first portion 426a of the needle channel 426 extends through each connection portion 425 of the undulations 422 of the second jaw member 420, and the second portion 426b of the needle channel 426 interconnects the second portion 426a of the needle channel 426 and each peak 423 of the second jaw member 420. The first portions 416a, 426a of the respective needle channels 416, 426 are configured to allow the respective needle 510, 520 to pass through. The second portions 416b, 426b of the respective needle channels 416, 426 are configured to allow the suture 600 to pass through (e.g., when the suture 600 is released from the needle assembly 500).

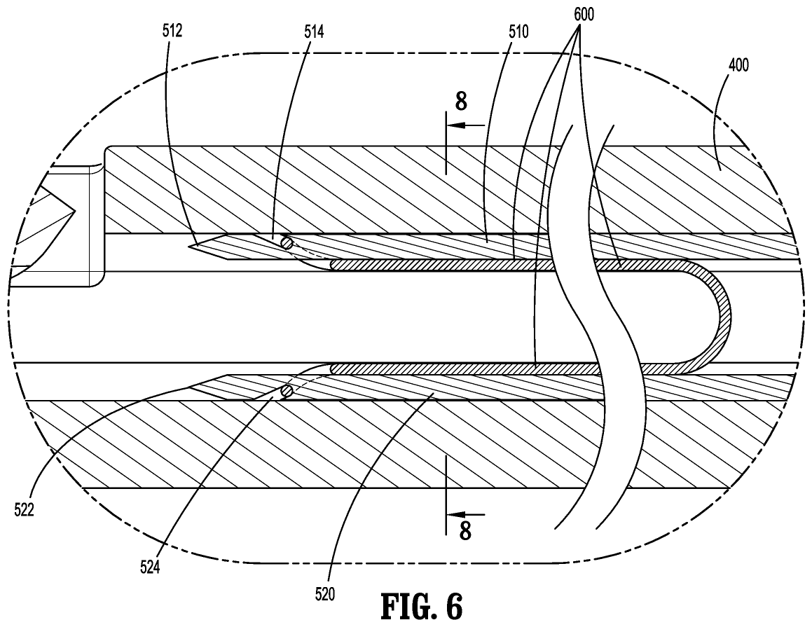

Additionally, as shown in FIGS. 6-8 and 10, the needle channels 416, 426 extend at least partially through a portion of the end effector 400 that is proximal of the jaw members 410, 420 (and/or proximally of the elongated portion 300 of the surgical instrument 100), such that a distal end 502 of the needle assembly 500 is positionable proximally of the jaw members 410, 420.

With particular reference to FIG. 4, the needle assembly 500 is shown. The needle assembly 500 includes a first needle 510, a second needle 520, and a connecting portion 530. The first needle 510 and the second needle 520 are connected to each other by the connecting portion 530. The elongated slide member 222 is mechanically engaged with at least one of the first needle 510, the second needle 520, and/or the connecting portion 530, such that longitudinal translation of the elongated slide member 222 results in a corresponding longitudinal translation of the needle assembly 500. The first needle 510 includes a point 512 at its distal end, and a suture catch 514 proximally adjacent its distal end. The second needle 520 includes a point 522 at its distal end, and a suture catch 524 proximally adjacent its distal end. The suture catches 514, 524 are notches or grooves in the respective first needle 510 and second needle 520 and are configured to releasably engage a suture 600. More particularly, a first suture loop 610 is configured to engage the suture catch 514 of the first needle 510, and a second suture loop 620 is configured to engage the suture catch 524 of the second needle 520. Thus, when the suture 600 is engaged with the needle assembly 500, distal translation of the needle assembly 500 results in a corresponding distal translation of the suture 600.

Referring now to FIGS. 5-11, in use, the jaw members 410, 420 of the surgical instrument 100 are positioned about tissue “T” while the jaw members 410, 420 are in the open position (FIG. 5). In this position, the needle assembly 500 is positioned proximally of the jaw members 410, 420 (FIGS. 5, 6 and 8).

Next, and with particular reference to FIGS. 7 and 9, the jaw members 410, 420 are moved to the approximated position (e.g., the first jaw member 410 pivots toward the second jaw member 420) and clamps the tissue “T” therebetween. As shown in FIG. 7, the tissue “T” fills or substantially fills spaces 417, 427 defined between adjacent undulations 412, 422, respectively. (Unfilled spaces 417, 427 are illustrated in FIG. 5.)

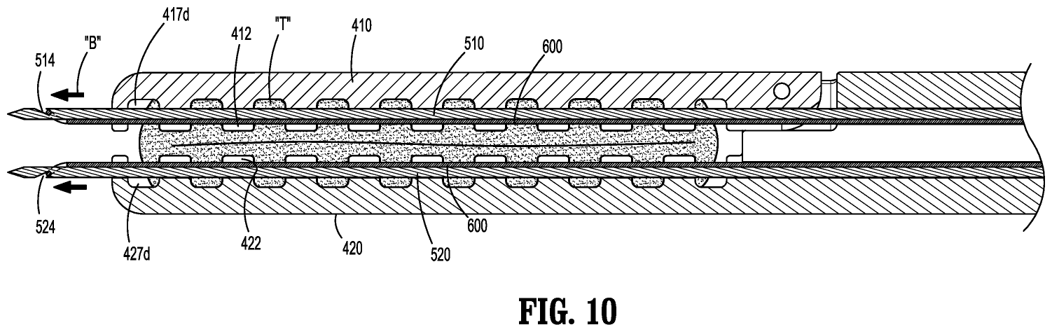

After the tissue “T” has been clamped, the needle assembly 500 is advanced distally, in the general direction of arrow “B” in FIG. 10. As the needle assembly 500 translates distally, the first needle 510 moves through the needle channel 416 of the first jaw member 410, and the second needle 520 moves through the needle channel 426 of the second jaw member 420. More particularly, as the first needle 510 is advanced distally through the needle channel 416 of the first jaw member 410, the point 512 of the first needle 510 punctures and extends through the tissue “T” within the spaces 417 between adjacent undulations 412 of the first jaw member 410. After the point 512 of the first needle 510 travels through the distal-most space 417d (FIG. 10), the point 512 travels distally past the first jaw member 410. Similarly, as the second needle 520 is advanced distally through the needle channel 426 of the second jaw member 420, the point 522 of the second needle 520 punctures and extends through the tissue “T” within the spaces 427 between adjacent undulations 422 of the second jaw member 420. After the point 522 of the second needle 520 travels through the distal-most space 427d (FIG. 10), the point 522 travels distally past the second jaw member 420.

Additionally, since the suture 600 is engaged with the first needle 510 and the second needle 520, the distal advancement of the first needle 510 and the second needle 520 causes the suture 600 to follow the same or similar path of the first needle 510 and the second needle 520 through the channels 416, 426 and tissue “T” within the spaces 417, 427 between adjacent undulations 412, 422 of the first jaw member 410 and the second jaw member 420, respectively. The suture 600 is released (e.g., in response to proximal movement of the needles 510, 520) through the second portions 416b, 426b of the respective needle channels 416, 426 (FIG. 9).

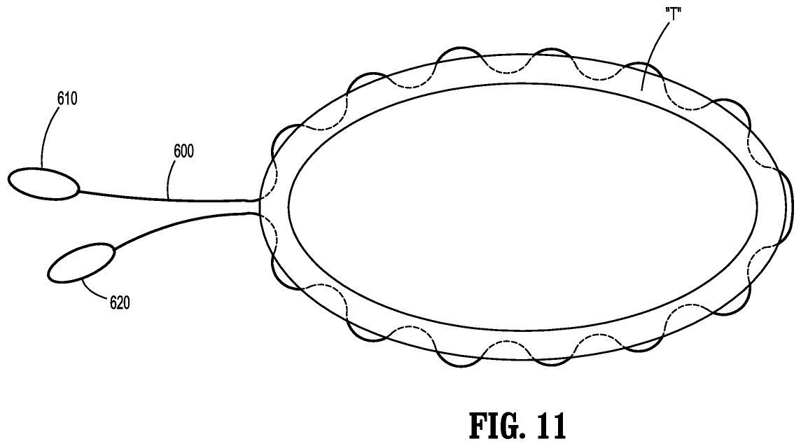

As a result of the distal translation of the first needle 510, the second needle 520, and the suture 600 through the tissue “T,” a purse string is formed around and through the tissue “T”, as schematically shown in FIG. 11.

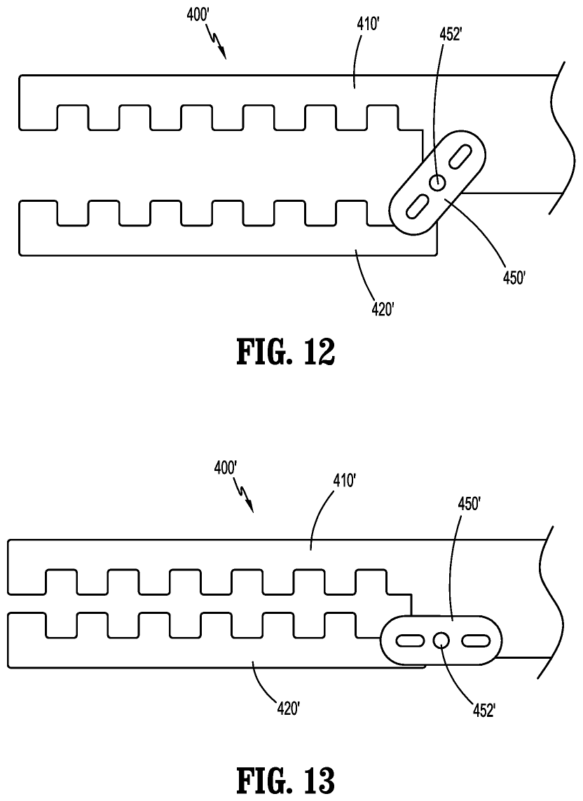

FIGS. 12 and 13 illustrates an instrument in accordance with another aspect of the present disclosure that includes an end effector 400′ having a first jaw member 410′ and a second jaw member 420′. As opposed to the pivotal relationship of the jaw members 410, 420 of the end effector 400, end effector 400′ includes a camming relationship between jaw members 410′, 420′. In particular, a cam member 450′ is pivotally engaged with a proximal portion of the first jaw member 410′ about a pivot pin 452′ and is coupled to a proximal portion of the second jaw member 420′, for instance. Further, a drive shaft (not explicitly shown in FIGS. 12 and 13) is operably coupled to a proximal portion of the cam member 450′. Accordingly, longitudinal translation of the drive shaft relative to the first jaw member 410′ causes the second jaw member 420′ to move toward the first jaw member 410′ between an open position (FIG. 12) and an approximated position (FIG. 13).

While the above description contains many specifics, these specifics should not be construed as limitations on the scope of the present disclosure, but merely as illustrations of various aspects thereof. Therefore, the above description should not be construed as limiting, but merely as exemplifications of various aspects. Those skilled in the art will envision other modifications within the scope and spirit of the claims appended hereto.

What is claimed is:

1. A surgical instrument comprising:
a handle assembly;
an elongated portion extending distally from the handle assembly and defining a longitudinal axis;
an end effector disposed adjacent a distal end of the elongated portion and including a first jaw member and a second jaw member, the first jaw member being movable relative to the second jaw member between a first position where the jaw members are in an open position, and a second position where the jaw members are in an approximated position, the first jaw member including:
a plurality of undulations, each undulation of the plurality of undulations including a peak, a valley and a connecting portion which interconnects one peak with one valley; and
a needle channel extending through each undulation of the plurality of undulations, the needle channel being parallel to the longitudinal axis;
the second jaw member including:
a plurality of undulations, each undulation of the plurality of undulations including a peak, a valley and a connecting portion which interconnects one peak with one valley; and
a needle channel extending through each undulation of the plurality of undulations, the needle channel being parallel to the longitudinal axis; and
a needle assembly including a first needle and a second needle, each of the first needle and the second needle including a suture catch configured to releasably engage a portion of a suture, the first needle being movable from a first position where a distal end of the first needle is positioned proximally of the first jaw member to a second position where the distal end of the first needle is positioned distally of the first jaw member, the second needle being movable from a first position where a distal end of the second needle is positioned proximally of the second jaw member to a second position where the distal end of the second needle is positioned distally of the second jaw member.

2. The surgical instrument according to claim 1, wherein the peaks of at least a majority of undulations of the plurality of undulations of the first jaw member are flat along a majority of their lengths in a direction that is parallel to the longitudinal axis.

3. The surgical instrument according to claim 2, wherein the peaks of at least a majority of undulations of the plurality of undulations of the second jaw member are flat along a majority of their lengths in a direction that is parallel to the longitudinal axis.

4. The surgical instrument according to claim 3, wherein the valleys of at least a majority of undulations of the plurality of undulations of the first jaw member are flat along a majority of their lengths in a direction that is parallel to the longitudinal axis.

5. The surgical instrument according to claim 4, wherein the valleys of at least a majority of undulations of the plurality of undulations of the second jaw member are flat along a majority of their lengths in a direction that is parallel to the longitudinal axis.

6. The surgical instrument according to claim 2, wherein a cumulative length of the flat portions of the peaks of the plurality of undulations of the first jaw member is between about 40% and about 60% of a total length of the first jaw member.

7. The surgical instrument according to claim 3, wherein a cumulative length of the flat portions of the peaks of the plurality of undulations of the first jaw member is between about 40% and about 60% of a total length of the first jaw member, and wherein a cumulative length of the flat portions of the peaks of the plurality of undulations of the second jaw member is between about 40% and about 60% of a total length of the second jaw member.

8. The surgical instrument according to claim 3, wherein the connecting portions of at least a majority the plurality of undulations of the first jaw member are perpendicular to the longitudinal axis.

9. The surgical instrument according to claim 1, wherein the needle channel of the first jaw member extends through the connecting portion of each undulation of the plurality of undulations of the first jaw member, and wherein the needle channel of the second jaw member extends through the connecting portion of each undulation of the plurality of undulations of the second jaw member.

10. The surgical instrument according to claim 1, wherein each undulation of the plurality of undulations of the first jaw member is longitudinally aligned with one undulation of the plurality of undulations of the second jaw member.

11. The surgical instrument according to claim 1, wherein the needle channel of the first jaw member defines a cross-sectional shape having a first portion and a second portion, the first portion of the cross-sectional shape is circular, and the second portion of the cross-sectional shape is rectangular.

12. The surgical instrument according to claim 11, wherein the first portion of the cross-sectional shape of the needle channel of the first jaw member extends through the connecting portion of each undulation of the plurality of undulations of the first jaw member.

13. The surgical instrument according to claim 12, wherein the second portion of the cross-sectional shape of the needle channel of the first jaw member extends through the peak of each undulation of the plurality of undulations of the first jaw member.

14. A surgical instrument comprising:
a handle assembly;
an elongated portion extending distally from the handle assembly and defining a longitudinal axis;
a first jaw member disposed adjacent a distal end of the elongated portion, the first jaw member including a plurality of undulations, each undulation including a peak, a valley, and a connecting portion which interconnects one peak with one valley; and
a needle channel extending through each undulation of the plurality of undulations and being parallel to the longitudinal axis, the needle channel defining a cross-section having a first portion and a second portion, the first portion extending through the connecting portion of each undulation of the plurality of undulations, the second portion extending through the peak of each undulation of the plurality of undulations.

15. The surgical instrument according to claim 14, wherein the first portion of the cross-section of the needle channel is circular, and the second portion of the cross-section of the needle channel is rectangular.

16. The surgical instrument according to claim 14, wherein the peaks of at least a majority of undulations of the plurality of undulations of the first jaw member are flat along a majority of their lengths in a direction that is parallel to the longitudinal axis, wherein the valleys of at least a majority of undulations of the plurality of undulations of the first jaw member are flat along a majority of their lengths in a direction that is parallel to the longitudinal axis, and wherein the connecting portions of at least a majority the plurality of undulations of the first jaw member are perpendicular to the longitudinal axis.

17. The surgical instrument according to claim 16, wherein a cumulative length of the flat portions of the peaks of the plurality of undulations of the first jaw member is between about 40% and about 60% of a total length of the first jaw member.

18. A method of performing a purse string closure, comprising:
positioning an end effector of a laparoscopic surgical instrument adjacent a surgical site;
moving a first jaw member of the end effector toward a second jaw member of the end effector to clamp tissue therebetween;
advancing a first needle and a suture through a plurality of undulations of the first jaw member such that the first needle punctures tissue disposed between adjacent undulations of the first jaw member; and
advancing a second needle and the suture through a plurality of undulation of the second jaw member such that the second needle punctures tissue disposed between adjacent undulations of the second jaw member.

19. The method according to claim 18, wherein advancing the first needle including moving the first needle from a first position where a distal tip of the first needle is disposed proximally of the first jaw member to a second position where the distal tip of the first needle is disposed distally of the first jaw member.

20. The method according to claim 18, wherein advancing the first needle and advancing the second needle occurs simultaneously.
